(19) United States
(12) Patent Application Publication
(10) Pub. No.: US 2025/0325581 A1
(43) Pub. Date: Oct. 23, 2025

(54) USE OF SODIUM PENTABORATE PENTAHYDRATE AS A CHEMOTHERAPEUTIC AGENT

(71) Applicant: YEDITEPE UNIVERSITESI, Atasehir/Istanbul (TR)

(72) Inventors: Fikrettin SAHIN, Atasehir/Istanbul (TR); Nezaket TURKEL SESLI, Atasehir/Istanbul (TR); Sidika TAPSIN GULLUOGLU, Kadikoy/Istanbul (TR)

(73) Assignee: YEDITEPE UNIVERSITESI, Atasehir/Istanbul (TR)

(21) Appl. No.: 18/864,037

(22) PCT Filed: Apr. 10, 2023
(86) PCT No.: PCT/TR2023/050333

§ 371 Date: Nov. 8, 2024

(30) Foreign Application Priority Data
May 9, 2022 (TR) 2022/007524

(51) Int. Cl. A61K 33/22; A61P 35/00

(52) U.S. Cl. CPC A61K 33/22; A61P 35/00

(57) ABSTRACT

The present invention relates to the use of sodium pentaborate pentahydrate enriched with boron isotope (¹⁰B, ¹¹B) as a chemotherapeutic agent thanks to its anticarcinogenic effect on renal cancer. As a result of the experimental studies performed within the scope of the invention, it has been shown that while the number of renal cancer cells decreased by 50%, this effect occurs by arresting the cell cycle and slowing down cell proliferation, therefore sodium pentaborate pentahydrate enriched with boron isotope can be used as a chemotherapeutic agent in cancer treatment.

### FIELD OF THE INVENTION

The present invention relates to the use of sodium pentaborate pentahydrate enriched with boron isotope (¹⁰B, ¹¹B) as a chemotherapeutic agent due to its anticarcinogenic effect on renal cancer cell lines.

### BACKGROUND OF THE INVENTION

The kidneys are a pair of organs which are located in the upper abdomen on the back and below both sides of the rib cage and produce urine. The produced urine is transferred to the urinary bladder through two thin tubes called ureters. The main function of the kidneys is to remove harmful substances produced by metabolic activities and excess water via urine. They also play a role in blood production and blood pressure regulation.

Renal cell cancer, which usually develops between the ages of 50-70 and is observed 3 times more often in men than in women, originates from the tissues in the kidney that filter blood and produce urine. While growing kidney cancer most commonly spreads to organs such as the large intestine, liver, and pancreas, it can also metastasize to distant organs. Many factors such as smoking, obesity, high blood pressure, and dialysis can increase the risk of developing renal cancer.

Although surgery is the standard method in the treatment of renal cancer, the location, size, metastasis of the tumor, the stage of the disease, the physical state of the patient (age, weight, etc.) are evaluated for the treatment, and one or more of the methods such as surgical treatment, radiotherapy, immunotherapy, and chemotherapy is/are used.

The use of chemotherapy to kill cancer cells has a very limited effect in kidney cancer, even though it is effective in many other cancers. This has made it crucial to develop an effective chemotherapeutic drug for the treatment of renal cancer. In addition to being used to reduce the size and metastasis of the tumor before surgery, chemotherapeutic method can also be used after surgery to kill any remaining cancer cells. It can also be used alone as an effective anti-carcinogenic agent.

In all the studies in the prior art, boric acid and phenylboronic acid forms of boron were used and its effect on renal cancer has not been previously studied. Within the scope of this application, sodium pentaborate pentahydrate derivative enriched with boron isotope (¹⁰B, ¹¹B) was used, its efficacy was increased and studies on its efficacy on renal cancer were performed. Within the scope of the patent, it is desired to provide protection for the selective anticarcinogenic effect of sodium pentaborate pentahydrate derivative enriched with boron isotope (¹⁰B, ¹¹B) on cancer cells and its use in single or combination treatment.

WO2020020086014 (Yeditepe University), as a known technical application, discloses the use of lead nanoparticles in the treatment of cancer. The boron derivative of the present patent is in salt form and the boron derivative in the said patent is a heavy metal compound. There are no similarities in terms of production technique and mechanisms of action.

### SUMMARY OF THE INVENTION

The objective of the invention is to reduce the number of renal cancer cells by 50% through the anticarcinogenic activity of sodium pentaborate pentahydrate (NaB) enriched with boron isotope (¹⁰B, ¹¹B) as a chemotherapeutic agent on renal cancer cells.

Another objective of the invention is using the sodium pentaborate pentahydrate (NaB) in the treatment of renal cancer by delaying the cell cycle and slowing down the cell proliferation in the cancer cells.

### DETAILED DESCRIPTION OF THE INVENTION

“THE USE OF SODIUM PENTABORATE PENTAHYDRATE AS A CHEMOTHERAPEUTIC AGENT,” which was performed to achieve the objective of the present invention is illustrated in the accompanying figures, in which:

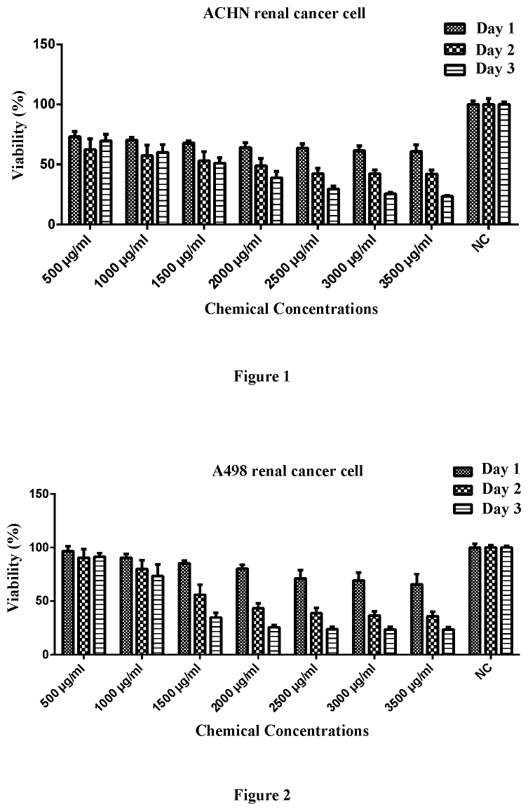

FIG. 1 is a graphical illustration of the effect of sodium pentaborate pentahydrate on the viability of ACHN renal cancer cells (colorimetric tetrazolium (MTS) viability assay).

FIG. 2 is a graphical illustration of the effect of sodium pentaborate pentahydrate on the viability of A498 renal cancer cells (colorimetric tetrazolium (MTS) viability assay).

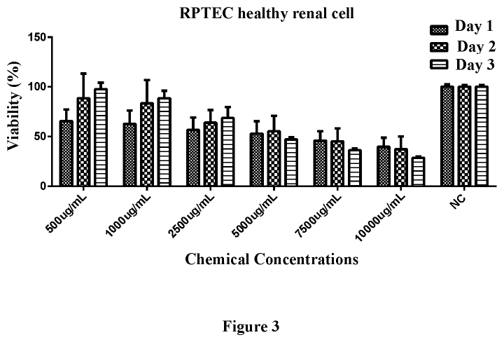

FIG. 3 is a graphical illustration of the effect of sodium pentaborate pentahydrate on the viability of RPTEC healthy renal cells (colorimetric tetrazolium (MTS) viability assay).

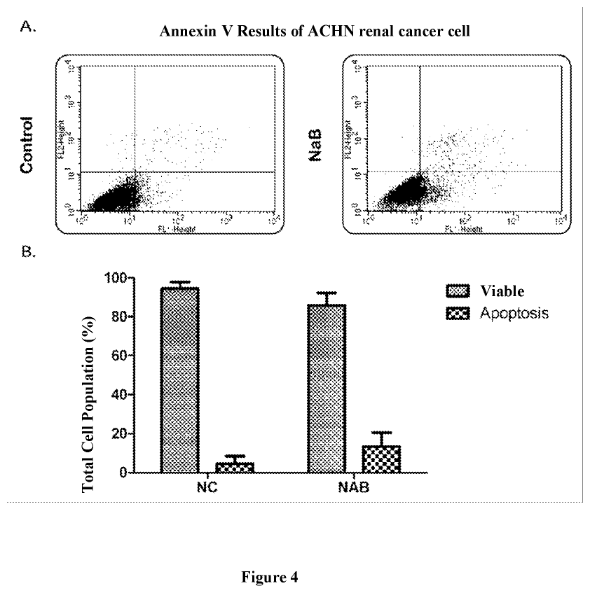

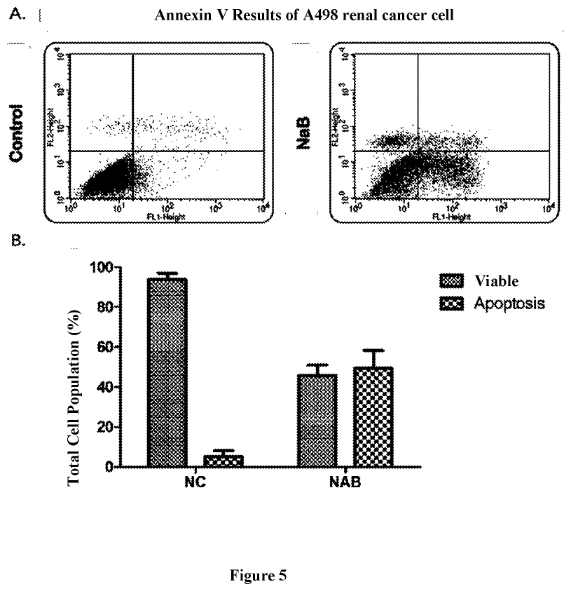

FIG. 4 is a graphical illustration of the effect of sodium pentaborate pentahydrate on the death of ACHN renal cancer cells via apoptosis (Annexin V assay).

FIG. 5 is a graphical illustration of the effect of sodium pentaborate pentahydrate on the death of A498 renal cancer cells via apoptosis (Annexin V assay).

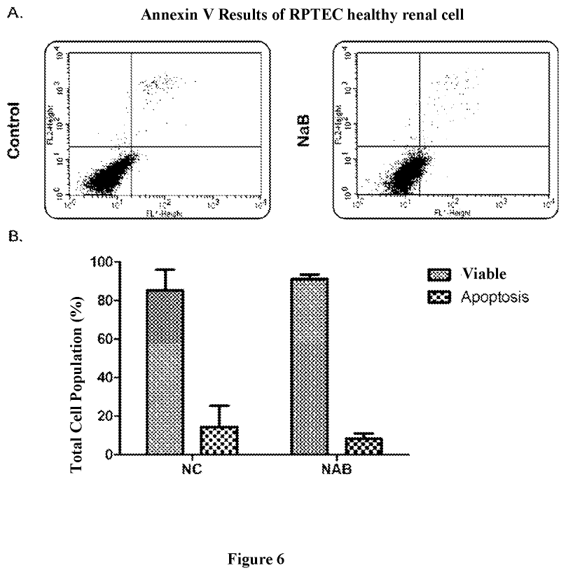

FIG. 6 is a graphical illustration of the effect of sodium pentaborate pentahydrate on the death of RPTEC healthy renal cells via apoptosis (Annexin V assay).

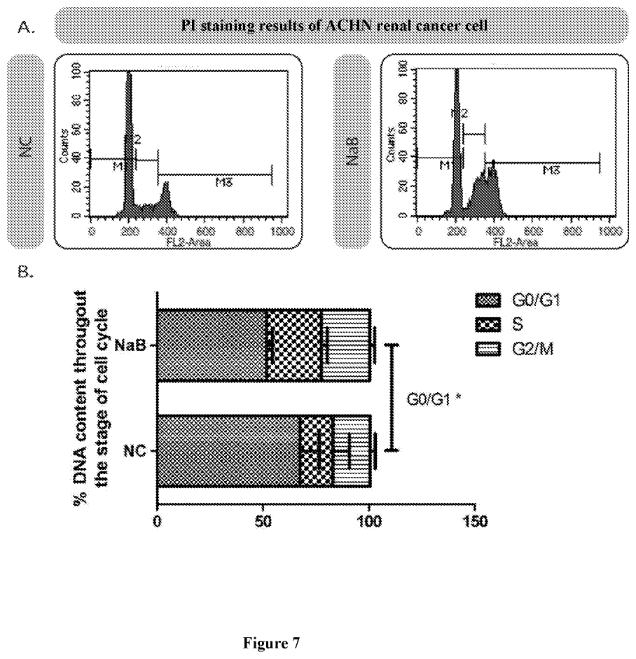

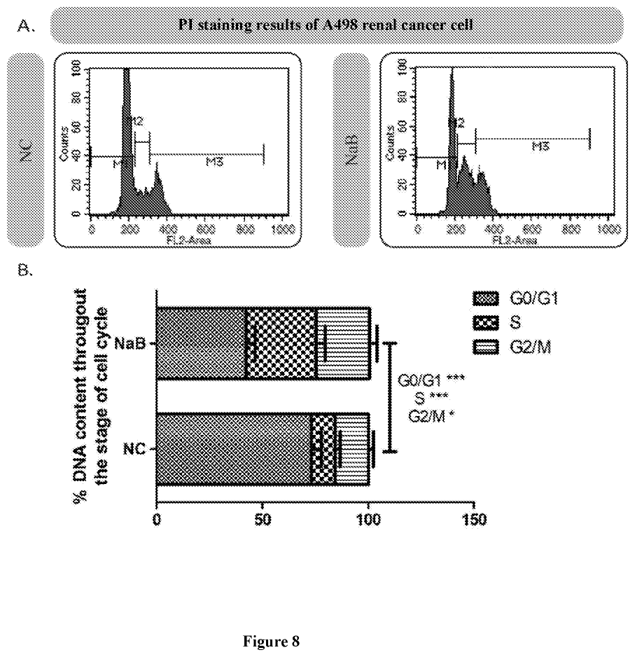

FIG. 7 is a graphical illustration of the effect of sodium pentaborate pentahydrate on cell division of ACHN renal cancer cells (Propidium Iodide staining) (* p<0.05).

FIG. 8 is a graphical illustration of the effect of sodium pentaborate pentahydrate on cell division of A498 renal cancer cells (Propidium Iodide staining) (* p<0.05, *** p<0.0001).

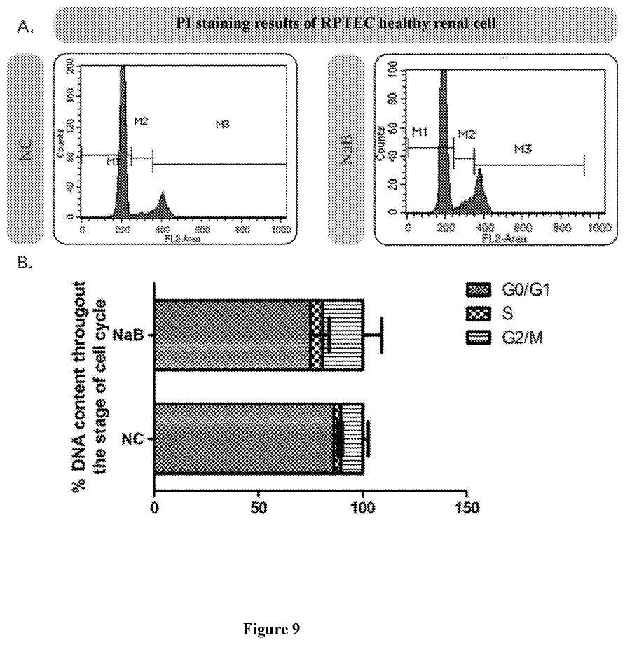

FIG. 9 is a graphical illustration of the effect of sodium pentaborate pentahydrate on cell division of RPTEC healthy renal cells (Propidium Iodide staining).

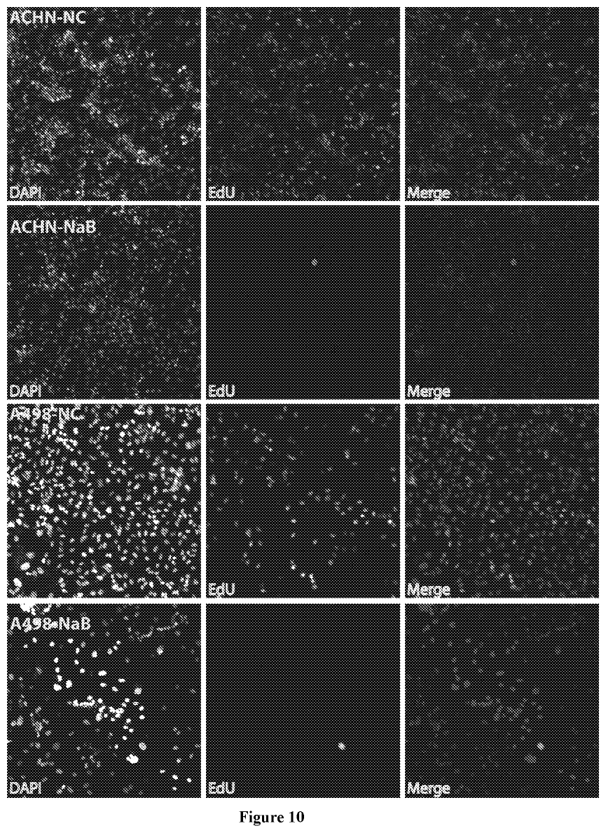

FIG. 10 is microscopic illustrations of the effect of sodium pentaborate pentahydrate on the proliferation of ACHN and A498 renal cancer cells (EdU staining, confocal microscopy image).

The present invention relates to the use of sodium pentaborate pentahydrate (NaB) enriched with boron isotope (¹⁰B, ¹¹B) as a chemotherapeutic agent for the treatment of cancer (renal cancer) through its anticarcinogenic activity on cancer cells (particularly renal cancer cells are discussed within the scope of the invention). Within the scope of experimental studies performed during the development of the present invention, it has been observed that sodium pentaborate pentahydrate (NaB) enriched with boron isotope (¹⁰B, ¹¹B) delays the cell cycle and slows down cell proliferation in cancer cells.

### EXPERIMENTAL STUDIES

In the experimental studies performed within the scope of the invention to determine the activity of sodium pentaborate pentahydrate (NaB) enriched with boron isotope (¹⁰B, ¹¹B) which is used as a chemotherapeutic agent within the scope of the invention, the following were carried out respectively:
weighing the powder sodium pentaborate pentahydrate at the desired concentration per milliliter, dissolving it in the medium in which the cells were grown, and sterilizing it by filtering through a 0.22 μm filter immediately before the start of the experiment so that the solutions will be fresh,
adding the solutions separately at ratios of 500 μg/ml, 1000 μg/ml, 1500 μg/ml, 2000 μg/ml, 2500 μg/ml, 3000 μg/ml, 3500 μg/ml for renal cancer cells (ACHN, A498), and 500 μg/ml, 1000 μg/ml, 2500 μg/ml, 5000 μg/ml, 7000 μg/ml for healthy renal cells (RPTEC),
incubating under conditions of 37° C., air ratios of 5% (v/v) CO₂ and 95% (v/v) from hour 0 until the end of hour 72,
performing colorimetric tetrazolium (MTS) viability assays at the end of hours 24, 48, and 72 (on Day 1, Day 2, and Day 3),
determining the half maximal inhibitory concentration (IC50) at the end of hour 72,
applying the IC50 value determined for cancer cells to healthy (RPTEC) and cancer cells (ACHN and A498) for 72 hours under the above-mentioned incubation conditions and evaluating apoptosis and cell cycle at the end of hour 72.

The chemotherapeutic agent of the present invention comprises sodium pentaborate pentahydrate and exhibits an apoptotic effect on renal cancer cells. Sodium pentaborate pentahydrate is a compound having a molecular weight of 295.107 grams/mol and the chemical formula of B₅H₁₀NaO₁₃. The effective dose of NaB on renal cancer was determined by colorimetric tetrazolium (MTS) viability assay on renal cancer cells (ACHN and A498) and healthy renal cells (RPTEC). MTS (3-(4,5-dimethyl-thiazol-2-yl)-5-(3-carboxy-methoxy-phenyl)-2-(4-sulfo-phenyl)-2H-tetrazolium) is a colorimetric assay based on the tetrazolium salt. The assay measures the enzyme activity which reduces MTS to the purple formazan. Cell cultures incubated for two hours are read by measuring the absorbance at 490 nm wavelength by means of ELISA. The NaB compound is dissolved at different concentrations (at ratios of 500 μg/ml, 1000 μg/ml, 1500 μg/ml, 2000 μg/ml, 2500 μg/ml, 3000 μg/ml, 3500 μg/ml for renal cancer cells, and 500 μg/ml, 1000 μg/ml, 2500 μg/ml, 5000 μg/ml, 7000 μg/ml for healthy renal cells) in the medium suitable for the cell used (30 seconds of vortexing is applied after the addition of the agent), filtered through 0.22 micron filters, and sterilized. MTS assays are performed at hours 24, 48, and 72, and as a result, the concentrations of dead cells at the IC50 dose are determined and the next steps are continued with these dosages. The doses which killed 50% of renal cancer cells (median TD) at hours 24, 48 and 72 are shown in Table 1 and FIGS. 1, 2, 3. As a result of the assay, the dose which killed 50% of renal cancer cells was determined as 1400 μg/ml (4.7 mM) and 1550 μg/ml (5.2 mM), respectively, for A498 and ACHN cell lines (FIGS. 1 and 2). For the RPTEC cell line, the dose of 1550 μg/ml, which is observed to be harmless to healthy cells and is the highest dose determined for cancer cells, was found to be appropriate for usage (FIG. 3).

TABLE 1
IC50s determined by the results of MTS
performed at hours 24, 48, and 72.

| | Hour 24 | Hour 48 | Hour 72 |
|---|---|---|---|
| ACHN | >7000 μg/ml | 2000 μg/ml | 1550 μg/ml |
| A498 | >7000 μg/ml | 1900 μg/ml | 1400 μg/ml |

Whether the death rates of renal cancer cells treated with the agent at the dosage determined by the MTS assay are dependent on apoptosis or not is determined by Annexin V assay. In normal cells, phosphatidylserine (PS), which is a type of lipid, is present on the surface of the cell membrane. During apoptosis, PS present in the cell membrane is translocated to the outer surface of the cell membrane. This translocation does not disrupt the integrity of the cell membrane. Annexin V binds to PSs, which were translocated to the outer surface of the cell, thereby making the apoptotic cell visible and it is measured by flow cytometry. Since Annexin V binding can also be observed on the surface of necrotic cells, Propidium Iodide (Red Fluorescence), which stains only dead cells, was added as a second stain. Cells, which are simultaneously stained with Annexin V-FITC (Green Fluorescence) and Propidium Iodide (Red Fluorescence), allow viable cells (FITC−PI−), early apoptotic cells (FITC+PI−), and late apoptotic or necrotic cells (FITC+PI+) to be distinguished from each other. Cells marked with Annexin V-FITCH-conjugated markers are analyzed by flow cytometry.

The phase of cell division the renal cancer cells treated with the agent at the dosage determined by the MTS assay is measured by flow cytometry following propidium iodide staining. Propidium iodide selectively binds between bases in nucleic acids, so that it becomes a mediator molecule between the bases and the fluorescent molecule. The assay is performed based on fluorescence intensity. For example, cells that are not dividing or are preparing to divide (i.e., in G1 phase) have 1 copy of DNA, thus fluorescing at 1× intensity, whereas cells in G2/M phase that are dividing have 2 copies of DNA, thus fluorescing at 2×.

Cell proliferation analyses of renal cancer cells treated with the agent at the dosage determined by the MTS assay are performed by EdU (Ethinyl-2′-deoxyuridine) assay. EdU stain binds to newly formed replicated DNA molecules during the DNA synthesis phase, enabling quantitative comparisons of cell populations in the DNA synthesis phase as a result of NaB treatments. For this assay, 10 uM EdU is applied to the cells cultured on slides placed in 6-well plates. Following the incubation, cells are fixed for 15 minutes with 3.7% formaldehyde diluted in PBS, and then washed with 3% BSA also diluted in PBS. Cell membrane permeability is increased by treatment with 0.5% nonionic surfactant diluted in PBS at room temperature for 20 minutes. After a final washing with 3% BSA solution, the reaction cocktail is added to the cells. Cells are incubated at room temperature for 30 minutes, and then the EdU-stained cells, which are actively synthesizing DNA, are observed under a confocal microscope.

In this study, the effect of sodium pentaborate pentahydrate (NaB) enriched with boron isotope (¹⁰B, ¹¹B) on renal cancer cells as a chemotherapeutic agent was investigated in a laboratory setting. Sodium pentaborate pentahydrate compound enriched with boron isotope (¹⁰B, ¹¹B) was prepared at different concentrations and applied to media containing healthy cells and cancer cells and its effects on cell viability, cell cycle, and proliferation were observed.

In line with the results obtained, it has been observed that it selectively leads cancer cells to programmed cell death (FIGS. 4-6), in addition to this effect, it arrests the cell cycle (FIGS. 7-9) and slows down cell proliferation (FIG. 10). Therefore, it has been shown that sodium pentaborate pentahydrate enriched with boron isotope (¹⁰B, ¹¹B) can be used as a chemotherapeutic agent in the treatment of carcinoma, sarcoma, leukemia, multiple myeloma, lymphoma, melanoma, brain and spinal cord tumors, reproductive cell tumors, and neuroendocrine tumors.

1. Sodium pentaborate pentahydrate (NaB) enriched with boron isotope (¹⁰B, ¹¹B) used as a chemotherapeutic agent for the treatment of cancer due to its anticarcinogenic activity.

2. Sodium pentaborate pentahydrate (NaB) enriched with a boron isotope (¹⁰B, ¹¹B) according to claim 1 as a chemotherapeutic agent for the treatment of renal cancer.
